(19) United States
(12) Patent Application Publication
(10) Pub. No.: US 2007/0173614 A1
(43) Pub. Date: Jul. 26, 2007

(54) Polyether composition

(75) Inventors: Keiichiro Mizuta, Hyogo (JP); Teruki Matsushita, Osaka (JP); Kenji Shimizu, Osaka (JP); Kazuo Takei, Osaka (JP); Michiyuki Kono, Osaka (JP); Manabu Kikuta, Kyoto (JP); Nobuo Ochiai, Shiga (JP)

Correspondence Address: ROBERT J JACOBSON PA, 650 BRIMHALL STREET SOUTH, ST PAUL, MN, 551161511, US

(73) Assignees: Nippon Shokubai Co., Ltd., Osaka-shi (JP); Dai-ichi Kogyo Seiyaku Co., Ltd., Kyoto-shi (JP)

(21) Appl. No.: 10/590,535

(22) PCT Filed: Mar. 8, 2005
(86) PCT No.: PCT/JP05/04404

§ 371 Date: Aug. 18, 2006

(30) Foreign Application Priority Data
Mar. 10, 2004 (JP) 2004-067900

(51) Int. Cl. C08G 65/32; C07C 213/02

(52) U.S. Cl. 525/403; 564/505

(57) ABSTRACT

An object of the present invention is to provide a polyether composition that can suppress reduction in a molecular weight of polyether having a low glass transition temperature and melting point. As a means of achieving this object, a polyether composition of the present invention contains polyether having a glass transition temperature of −50° C. or lower and a melting point of 55° C. or lower and, at the same time, contains at least one kind selected from the group consisting of compounds represented by the specified three general formulas.

### TECHNICAL FIELD

The present invention relates to a polyether composition containing polyether having a relatively low glass transition temperature and melting point.

### BACKGROUND ART

Previously, polyether has been widely used in various utilities, and since, particular, polyether having a low glass transition temperature and melting point has an advantage that it can be molded at a low temperature, and exerts excellent adherability, it is known that it is suitable in utilities such as an extrusion molding material, an antistatic agent, a polymer electrolyte, and a protecting membrane for a color filter.

However, polyether has a defect that it is easily oxidized and degraded due to its structure and, previously, when used in the aforementioned various utilities, a molecular weight is reduced in steps of storage, transport, molding, film forming, and the like, depending on the condition, in some cases. When a molecular weight is reduced like this, a problem such as difficulty in molding and film forming, reduction in physical properties such as a mechanical strength, and production of a smell substance and a toxic substance arises. For this reason, when polyether is handled in steps of storage, transport, molding, film forming, and the like, it is necessary to take a measure so as not to reduce a molecular weight, or performance at a relatively low temperature, or under the inert gas atmosphere.

However, in polyether having a relatively low glass transition temperature and melting point, specifically, polyether having a glass transition temperature of −50° C. or lower, and a melting point of 55° C. or lower, in the existing circumstances, reduction in a molecular weight occurs very easily, and reduction in a molecular weight can not be sufficiently suppressed only by taking the aforementioned measure so as not to reduce a molecular weight.

### DISCLOSURE OF THE INVENTION

### Object of the Invention

Then, an object of the present invention is to provide a polyether composition which can suppress reduction in a molecular weight of polyether having a low glass transition temperature and melting point.

### SUMMARY OF THE INVENTION

In order to solve the aforementioned problems, the present inventors intensively studied. As a result, a specified compound which can effectively suppress reduction in a molecular weight of polyol having a glass transition temperature of −50° C. or lower and a melting point of 55° C. or lower by incorporating into the polymer was found out. The present invention was completed based on this finding.

That is, the polyether composition of the present invention contains polyether having a glass transition temperature of −50° C. or lower, and a melting point of 55° C. or lower and, at the same time, also contains at least one kind selected from the group consisting of a compound represented by the following general formula (1), a compound represented by the following general formula (2) and a compound represented by the following general formula (3). 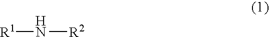
(in the formula (1), R¹ and R² are each independently an allyl group of a carbon number of 1 to 8, an alkenyl group, an aryl group, an alkoxyl group, or a substituted aryl group, or R¹ and R² may be bound to each other directly or via at least one kind element selected from C, O, S, P and N) 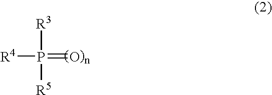
(in the formula (2), R³, R⁴ and R⁵ are each independently an alkyl group of a carbon number of 1 to 8, an alkenyl group, an aryl group, an alkoxyl group, a substituted aryl group, or an amino group, or R³ and R⁴ may be bound to each other directly or via at least one kind element selected from C, O, S, P and N, and n is 0 or 1) 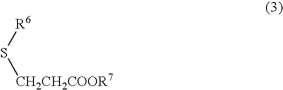
(in the formula (3), R⁶ is an organic residue, and R⁷ is an alkyl group of a carbon number of 1 to 30)

### EFFECTS OF THE INVENTION

The polyether composition of the present invention can effectively suppress reduction in a molecular weight of even polyether having a low glass transition temperature and melting point, and can avoid a problem such as difficulty in molding or film forming, reduction in physical properties such as a melting point and a mechanical strength, and production of a smell substance and a toxic substance.

### DETAILED DESCRIPTION OF THE INVENTION

The polyether composition of the present invention will be explained in detail below, but a scope of the present invention is not restricted by the explanation, and alternation can be appropriately practiced in such the range that the gist of the present invention is not deteriorated, also regarding aspects other than the following embodiments.

The polyether composition of the present invention contains, as a main component, polyether having a glass transition temperature of −50° C. or lower, and a melting point of 55° C. or lower. By a glass transition temperature of the polyether of −50° C. or lower, and a melting point of the polyether of 55° C. or lower, a composition having an advantage that molding at a low temperature becomes possible, and excellent adherability is exerted, is obtained. Further, for example, when the polyether composition of the present invention is used by formulating into a film, there is also an advantage that a film having better shape retaining force, flexibility, toughness and strength at its use temperature region can be obtained. A lower limit of a glass transition temperature and a melting point of the polyether is not particularly limited, but in particular a melting point is preferably 30° C. or higher, more preferably 35° C. or higher. When a melting point of the polyether is lower than 30° C., in the case where the polyether composition of the present invention is used by formulating into a film, there is a tendency that a film shape is retained with difficulty in its use temperature region. The glass transition temperature and the melting point in the present invention can be measured using a differential thermal analyzer, specifically, by rapidly heating a polymer from which volatile matters have been removed if necessary, to 80° C. and what has been moisture-conditioned to be melt once, cooling the polymer from 80° C. to −100° C. at 5° C./min to crystallize it, and elevating a temperature of the crystallized polymer from −100° C. to 80° C. at 5° C./min.

Preferably examples of the polyether include a polymer having a constitutional unit represented by the following general formula (4). 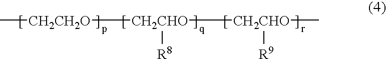
(in the formula (4), R⁸ and R⁹ are each independently an alkyl group of a carbon number of 1 to 8, an alkenyl group, an aryl group, an alkoxyl group, a substituted aryl group, or a group represented by the following general formula (5), all of p, q and r are an integer of 0 or larger, and p+q+r>0) 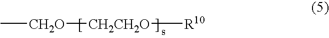
(in the formula (5), R¹⁰ is an alkyl group of a carbon number of 1 to 8, an alkenyl group, an aryl group, an alkoxyl group, or a substituted aryl group, and s is an integer of 0 or larger)

In the general formulas (4) and (5), specific examples of the alkyl group represented by R⁸, R⁹ and R¹⁰ include a methyl group, an ethyl group, a propyl group, and a butyl group, specific examples of the alkenyl group include an alkyl group, and a vinyl group, specific examples of the aryl group include a phenyl group, and a diphenyl group, specific examples of the alkoxyl group include a methoxy group, an ethoxy group, and a butoxy group, and specific examples of the substituted aryl group include a methylphenyl group, and an ethylphenyl group.

Specific examples of the polymer having a constitutional unit represented by the general formula (4) include polyethylene oxide, polypropylene oxide, polybutylene oxide, polyallyl glycidyl ether, polymethoxyethyl glycidyl ether, and a copolymer of monomers constituting these polymers (e.g. ethylene oxide/propylene oxide copolymer). The polyether may be one kind, or two or more kinds.

The polyether has an elongation viscosity under a shear rate of 100 to 500 (1/sec) of preferably 100 to 1,000,000 Pa·s, more preferably 500 to 500,000 Pa·s, further preferably 1,000 to 100,000 Pa·s. When the elongation viscosity is less than 100 Pa·s, there is a tendency that a strength when formulated into a film becomes insufficient. For example, when the polyether composition of the present invention is used by formulating into a film, since a tension of a film at melting becomes low upon molding into a film, there is a possibility that the film is cut at melting. On the other hand, when the elongation viscosity exceeds 1,000,000 Pa·s, there is a tendency that moldability is deteriorated. For example, when the polyether composition of the present invention is used by formulating into a film, since a torque becomes high upon extrusion into a film, and extrusion may become difficult, both cases are not preferable. By both of its melting point and elongation viscosity satisfying the aforementioned ranges, when the polyether composition of the present invention is used by formulating, for example, into a film, the polyether can combine a tension of a film at melting with a torque upon extrusion when molded into a film. And, a film better in shape retaining force, flexibility, toughness and strength at its use temperature region can be easily obtain. The elongation viscosity in the present invention is measured at a temperature of 100 to 110° C. under a resin sold matter of 100%, and measurement of the elongation viscosity can be specifically performed, for example, under the following conditions.

Measuring equipment: Twin Capillary Rheometer (“RH7-2 type” manufactured by Rossant)

Die: a long die (length 32 mm) short die (length 0.25 mm) having a diameter of 2 mm

Die angle: 180°

Retention time: 10 min

Atmosphere: dry air

Polymer pretreatment: a polymer is allowed to stand in a glove box at room temperature for 24 hours under the nitrogen atmosphere.

A weight average molecular weight (Mw) of the polyether may be appropriately set depending on utilities, is not particularly limited, but is preferably 10,000 to 1,500,000. When a weight average molecular weight is less than 10,000, there is a possibility that tack is produced upon molding and film forming, and handling property is deteriorated. On the other hand, when a weight average molecular weight exceeds 1,500,000, there is a possibility that molding or film forming themselves become difficult, and processibility is reduced.

A molecular weight distribution (Mw/Mn) of the polyether may be approximately set depending on utilities, is not particularly limited, but is preferably 5 or smaller. When a molecular weight distribution exceeds 5, there is a possibility that tack occurs upon molding or film forming, and handling property is deteriorated.

The polyether composition of the present invention also contains at least one kind selected from the group consisting of a compound represented by the general formula (1), a compound represented by the general formula (2) and a compound represented by the general formula (3) (hereinafter, referred to as “specified compound X” in some cases). Thereby, in the case of even the aforementioned polyether having a low glass transition temperature and melting point, reduction in its molecular weight can be effectively suppressed. The specified compound X may be any kind of a compound represented by the general formula (1), a compound represented by the general formula (2), and a compound represented by the general formula (3), and preferable is two or more kinds.

In the general formula (1), specific examples of the alkyl group represented by R¹ and R² include a methyl group, an ethyl group, a butyl group, a hexyl group, and an octyl group, specific examples of the alkenyl group include allyl group, a vinyl group, an ethenyl group, and a butenyl group, specific examples of the aryl group include a phenyl group, a diphenyl group, and a naphthyl group, specific examples of the alkoxyl group include a methoxy group, an ethoxy group, and a methoxyethyl group, and specific examples of the substituted aryl group include a methylphenyl group, and a methoxyphenyl group.

Specific examples of the compound represented by the general formula (1) include diphenylamines, dinaphthylamines, diphenylphenylenediamines, and phenothiazines. These may be only one kind, or two or more kinds.

As the compound represented by the general formula (1), in particular, diphenylamines represented by the general formula (6) are preferable. 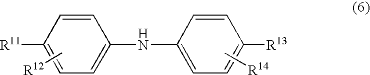
(in the formula (6), R¹¹, R¹², R¹³ and R¹⁴ are each independently a hydrogen atom or a hydrocarbon group of a carbon number of 1 to 18)

In the general formula (6), examples of the hydrocarbon group represented by R¹¹, R¹², R¹³ and R¹⁴ include a straight or branched alkyl group of a carbon number of 1 to 18, a straight or branched alkenyl group of a carbon number of 2 to 18, a cycloalkyl group of a carbon number of 6 to 18, and an aryl group of a carbon number of 6 to 18, and an aryl group may contain an allyl group or an alkenyl group having a carbon number of 1 to 12. Inter alia, an alkyl group such as a methyl group, an ethyl group, a propyl group, a butyl group, a tertiary butyl group, a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, and an isomer thereof is preferable, and an alkyl group of a carbon number of 3 to 10 is particularly preferable.

Specific examples of diphenylamines represented by the general formula (6) include diphenylamine, p,p′-dibutyldiphenylamine, p,p′-di-tertiary butyldiphenylamine, p,p′-dipentyldiphenylamine, p,p′-dihexyldiphenylamine, p,p′-diheptyldiphenylamine, p,p′-dioctyldiphenylamine, p,p′-dinonyldiphenylamine, monooctyldiphenylamine, monononyldiphenylamine, tetrabutyldiphenylamine, tetrahexyldiphenylamine, tetraoctyldiphenylamine, tetranonyldiphenylamine, mixed alkyldiphenylamine of a carbon number of 4 to 9, N-phenyl-1-naphthylamine, N-phenyl-2-naphthylamine, 4-n-butylaminophenol, 4-butyrylaminophenol, 4-nonanoylaminophenol, 4-dodecanoylaminophenol, 4-octadecanoylaminophenol, di(4-methoxyphenyl)amine, 2,6-di-tertiary-butyl-4-dimethylaminomethylphenol, 2,4′-diaminodiphenylmethane, 4,4′-diaminodiphenylmethane, N,N,N′,N′-tetramethyl-4,4′-diaminodiphenylmethane, 1,2-di[(2-methylphenyl)amino]ethane, 1,2-di(phenylamino)propane, (o-tolyl)niguanide, di[4-(1′,3′-dimethylbutyl)phenyl]amine, tertiary octylated N-phenyl-1-naphthylamine, and styrenated diphenylamine. Among them, in particular, phenothiazine, 10-methylphenothiazine, 2-methylphenothiazine, 2-trifluoromethylphenothiazine, and phenozadine are more preferable.

In the general formula (2), specific examples of the allyl group represented by R³, R⁴ and R⁵ include a methyl group, an ethyl group, a propyl group, and a butyl group, specific examples of the alkenyl group include an allyl group, a vinyl group, an ethenyl group, and a butenyl group, specific examples of the aryl group include a phenyl group, a diphenyl group, and a naphthyl group, specific examples of the alkoxyl group include a methoxy group, an ethoxy group, a phenoxy group, and a substituted phenoxy group, specific examples of the substituted aryl group include a methylphenyl group, and a methoxyphenyl group, and specific examples of the amino group include a methylamino group and a dimethylamino group.

Specific examples of the compound represented by the general formula (2) include triphenyl phosphite, tris(2,4-di-tertiary butylphenyl)phosphite, tris(2,5-di-tertiary butylphenyl)phosphite, tris(nonylphenyl)phosphite, tris(dinonylphenyl)phosphite, tris(mono-di-mixed nonylphenyl)phosphite, diphenylacid phosphite, 2,2′-methylenebis(4,6-di-tertiary butylphenyl)octyl phosphite, diphenyldecyl phosphite, diphenyloctyl phosphite, di(nonylphenyl)pentaerythritol diphosphite, phenyldiisodecyl phosphite, tributyl phosphite, tris(2-ethylhexyl)phosphite, tridecyl phosphite, trilauryl phosphite, dibutylacid phosphite, dilaurylacid phosphite, trilauryl trithiophosphite, bis(neopentyl glycol)-1,4-cyclohexanedimethyl diphosphite, bis(2,4-di-tertiary butylphenyl)pentaerythritol diphosphite, bis(2,5-di-tertiary butylphenyl)pentaerythritol diphosphite, bis(2,6-di-tertiary butyl-4-methylphenyl)pentaerythritol diphosphite, bis(2,4-dicumylphenyl)pentaerythritol diphosphite, distearylpentaerythritol diphosphite, tetra(C12 to 15 mixed alkyl)-4,4′-isopropylidenediphenyl phosphite, bis[2,2′-methylenebis(4,6-diaminophenyl)]isopropylidenediphenyl phosphite, tetratridecyl-4,4′-butylidenebis(2-tertiary butyl-5-methylphenol) diphosphite, hexa(tridecyl)-1,1,3-tris(2-methyl-5-tertiary butyl-4-hydroxyphenyl)butane-triphosphite, tetrakis(2,4-di-tertiary butylphenyl)biphenylene diphosphonite, tris(2-[(2,4,7,9-tetrakis tertiary butyldibenzo[d,f][1,3,2]dioxaphosphepin-6-yl)oxy]ethyl)amine, 9,10-dihydro-9-oxa-10-phosphaphenanthrene-10-oxide, 2-butyl-2-ethylpropanediol-2,4,6-tri tertiary butylphenol monophosphite, tetrakis(2-tertiary butyl-4-methylphenyl)biphenylene diphosphonite, tetrakis(2,4-di-tertiary amylphenyl)biphenylene diphosphonite, tetrakis(2,4-di-tertiary butyl-5-methylphenyl)biphenylene diphosphonite, and tetrakis(2-tertiary butyl-4,6-dimethylphenyl)biphenylene diphosphonite. These may be only one kind, or two or more kinds.

In the general formula (3), specific examples of the organic residue represented by R⁶ include an amino group, an imino group, an amido group, an ether group, an ester group, a hydroxyl group, a carboxyl group, a carbamoyl group, a cyano group, and a sulfide group, and specific examples of the alkyl group represented by R⁷ include a hexyl group, an octyl group, a lauryl group and a stearyl group.

Specific examples of the compound represented by the general formula (3) include dialkyl thiodipropionates such as dilauryl ester, dimyristyl ester, myristylstearyl ester, and distearyl ester of thiodipropionic acid; and β-alkylmercaptopropionic acid esters of polyol such as pentaerythritol tetra(β-dodecylmercaptopropionate). These may be only one kind, or two or more kinds.

A ratio of the specified compound X occupied in the polyether composition of the present invention is not particularly limited, but is preferably 0.001 to 20 parts by weight, more preferably 0.01 to 10 parts by weight, further preferably 0.05 to 5 parts by weight relative to 100 parts by weight of the polyether. When the specified compound X is too smaller than the aforementioned range, there is a possibility that reduction in a molecular weight of polyether can not be sufficiently suppressed. On the other hand, when the specified compound X is too more than the aforementioned range, there is a possibility that remarkable coloration occurs in the composition and, moreover, effect of suppressing reduction in a molecular weight is not improved to such an extent that it corresponds to increase to in an amount of a specified compound X, leading to high cost of the composition.

The polyether composition of the present invention may contain a phenol-based antioxidant agent which has previously been generally used for preventing oxidation, in addition to the polyether and the specified compound X.

Examples of the phenol-based antioxidant agent include 2,6-di-tertiary butyl-p-cresol, 2,6-diphenyl-4-octadecyloxyphenol, stearyl(3,5-di-tertiary butyl-4-hydroxyphenyl)propionate, distearyl(3,5-di-tertiary butyl-4-hydroxybenzyl)phosphonate, tridecyl-3,5-di-tertiary butyl-4-hydroxybenzyl thioacetate, thiodiethylenebis[(3,5-di-tertiary butyl-4-hydroxyphenyl)propionate], 4,4′-thiobis(6-tertiary butyl-m-cresol), 2-octylthio-4,6-di(3,5-di-tertiary butyl-4-hydroxyphenoxy)-s-triazine, 2,2′-methylenebis(4-methyl-6-tertiary butylphenol), bis[3,3-bis(4-hydroxy-3-tertiary butylphenyl)butyric acid]glycol ester, 4,4′-butylidenebis(2,6-di-tertiary butylphenol), 4,4′-butylidenebis(6-di-tertiary butyl-3-methylphenol), 2,2′-ethylidenebis(4,6-di-tertiary butylphenol), 1,1,3-tris(2-methyl-4-hydroxy-5-tertiary butylphenyl)butane, bis[2-tertiary butyl-4-methyl-6-(2-hydroxy-3-tertiary butyl-5-methylbenzyl)phenyl]terephthalate, 1,3,5-tris(2,6-dimethyl-3-hydroxy-4-tertiary butylbenzyl)isocyanurate, 1,3,5-tris(3,5-di-tertiary butyl-4-hydroxybenzyl)isocyanurate, 1,3,5-tris(3,5-di-tertiary butyl-4-hydroxybenzyl)-2,4,6-trimethylbenzene, 1,3,5-tris [(3,5-di-tertiary butyl-4-hydroxyphenyl)propyonyloxyethyl]isocyanurate, tetrakis[methylene-3-(3′,5′-di-tertiary butyl-4′-hydroxyphenyl)propionate]methane, 2-tertiary butyl-4-methyl-6-(2-acryloyloxy-3-tertiary butyl-5-methylbenzyl)phenol, 3,9-bis[2-(3-tertiary butyl-4-hydroxy-5-methylhydrocinnamoyloxy)-1,1-dimethylethyl]-2,4,8,10-tetraoxaspyro[5.5]undecane, and triethylene glycol bis[β-(3-tertiary butyl-4-hydroxy-5-methylphenyl)propionate]. These may be only one kind, or two or more kinds.

When the polyether composition of the present invention also contains the phenol-based antioxidant, a ratio to be contained is preferably 10 parts by weight or less relative to 100 parts by weight of the polyether. When the phenol-based antioxidant is too much, there is a tendency that remarkable coloration occurs in the composition and, moreover, there is a possibility that the cost of the composition becomes high.

The polyether composition of the present invention may appropriately contain, if necessary, further an additive such as a solvent, an aging preventing agent, a light stabilizer, a lubricant, a reinforcing agent, and a filler in such a range that the effect of the invention is not deteriorated.

### DETAILED DESCRIPTION OF THE PREFERRED EMBODIMENTS

The present invention will be explained more specifically below by way of Examples and Comparative Examples, but the present invention is not limited by them. Hereinafter, unless otherwise indicated, “% by weight” is expressed by “%”.

### EXAMPLE 1

2 g of an ethylene oxide/propylene oxide copolymer (“CP1000L” manufactured by SUMITOMO SEIKA CHEMICALS CO., LTD.; ethylene oxide/propylene oxide=90/10 mole %, weight average molecular weight 1,070,000, glass transition temperature −60° C., melting point 45° C., elongation viscosity under shear rate of 350 (1/sec) 58,000 Pa·s) which is polyether, 10 mg of styrenated diphenylamine (“Stearer LAS” manufactured by SEIKO CHEMICAL CO., LTD.) as a specified compound X, and 40 g of toluene as a solvent were placed into a reactor, and the mixture was stirred at 22° C. for 1 hour to obtain a polyether composition.

The resulting polyether composition was assessed as follows: that is, the resulting polyether composition was placed into a drier (air atmosphere) at 150° C., a weight average molecular weight (Mw) was measured after 2 hours (measuring apparatus: “LC-10AD” manufactured by Shimadzu Corporation, eluent: dimethylformamide solution containing 0.1% by weight of lithium bromide, standard polystyrene), and a molecular weight retaining rate relative to an initial weight average molecular weight (Mw₀: 1,070,000) of the used ethylene oxide/propylene oxide copolymer was calculated by the following equation. The results are shown in Table 1.
Molecular weight retaining rate (%)=(Mw/Mw₀)×100

### EXAMPLES 2 TO 4, AND COMPARATIVE EXAMPLE 1

According to the same manner as that of Example 1 except that an amount shown in Table 1 of a compound shown in Table 1 was used in place of the specified compound X used in Example 1, a polyether composition was obtained.

The resulting polyether composition was assessed as in Example 1. The results are shown in Table 1.

In Table 1, following abbreviations were used:

Stearer: styrenated diphenylamine (“Stearer LAS” manufactured by SEIKO CHEMICAL CO., LTD.)

PEP: bis(2,4-di-tertiary butylphenyl)pentaerythritol diphosphite (“PEP-24Q” manufactured by Asahi Denka Co., Ltd.)

TPS: dilauryl thiodipropionate (“SumilizerTPS” manufactured by Sumitomo Chemical Co., Ltd.)

TABLE 1

| | Specified compound X | | Molecular |
|---|---|---|---|
| | Kind | Amount (mg) | weight retaining rate (%) |
| Example 1 | Stearer | 10 | 81.4 |
| Example 2 | Phenothiazine | 10 | 82.0 |
| Example 3 | Stearer/PEP | 5/5 | 76.6 |
| Example 4 | Stearer/TPS | 5/5 | 72.0 |
| Comparative | None | 0 | 49.1 |
| Example 1 | | | |

### INDUSTRIAL APPLICATION

The polyether composition of the present invention is suitably used as an extrusion molding material, an antistatic agent, a polymer electrolyte, or a protecting membrane for a color filter.

1. A polyether composition, which comprises polyether having a glass transition temperature of −50° C. or lower and a melting point of 55° C. or lower, and at least one kind selected from the group consisting of a compound represented by the following general formula (1), a compound represented by the following general formula (2) and a compound represented by the following general formula (3). 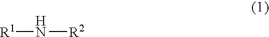
(in the formula (1), R¹ and R² are each independently an alkyl group of a carbon number of 1 to 8, an alkenyl group, an aryl group, an alkoxyl group or a substituted aryl group, and R¹ and R² may be bound to each other directly or via at least one kind element selected from C, O, S, P and N) 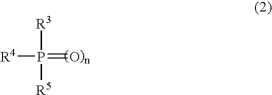
(in the formula (2), R³, R⁴ and R⁵ are each independently an alkyl group of a carbon number of 1 to 8, an alkenyl group, an aryl group, an alkoxyl group, a substituted aryl group, or an amino group, R³ and R⁴ may be bound to each other directly or via at least one kind element selected from C, O, S, P and N, and n is 0 or 1) 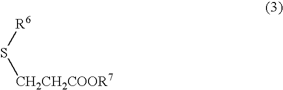
(In the formula (3), R⁶ is an organic residue, and R⁷ is an alkyl group of a carbon number of 1 to 30)

2. The polyether composition according to claim 1, wherein the polyether has an elongation viscosity under a shear rate of 100 to 500 (1/sec) of 100 to 1,000,000 Pa·s.
